(19) United States
(12) Patent Application Publication
(10) Pub. No.: US 2014/0200509 A1
(43) Pub. Date: Jul. 17, 2014

(54) Dissolvable Microneedles Comprising One Or More Encapsulated Cosmetic Ingredients

(71) Applicants: Issac David Cohen, Brooklyn, NY (US); Daniela Bratescu, Northport, NY (US); E. Althea Knight, Westbury, NY (US); Thomas Mammone, Farmingdale, NY (US)

(72) Inventors: Issac David Cohen, Brooklyn, NY (US); Daniela Bratescu, Northport, NY (US); E. Althea Knight, Westbury, NY (US); Thomas Mammone, Farmingdale, NY (US)

(21) Appl. No.: 13/924,142

(22) Filed: Jun. 21, 2013

Related U.S. Application Data
(60) Provisional application No. 61/665,974, filed on Jun. 29, 2012.

(51) Int. Cl. A61M 37/00

(52) U.S. Cl. CPC A61M 37/0015
USPC 604/46

(57) ABSTRACT

An applicator for applying cosmetic agents into human skin, comprising: (a) a base, (b) a plurality of dissolvable microneedles fixed to said base and projecting therefrom a distance sufficient to penetrate into the skin, said microneedles being made of a material that is capable of disintegration and dispersion into the skin, (c) a cosmetic agent carried by said microneedles for delivery by said microneedles into the skin, and (d) wherein said dissolvable microneedles are configured as a dissolvable exterior shell and said shell encapsulates one or more cosmetic agents.

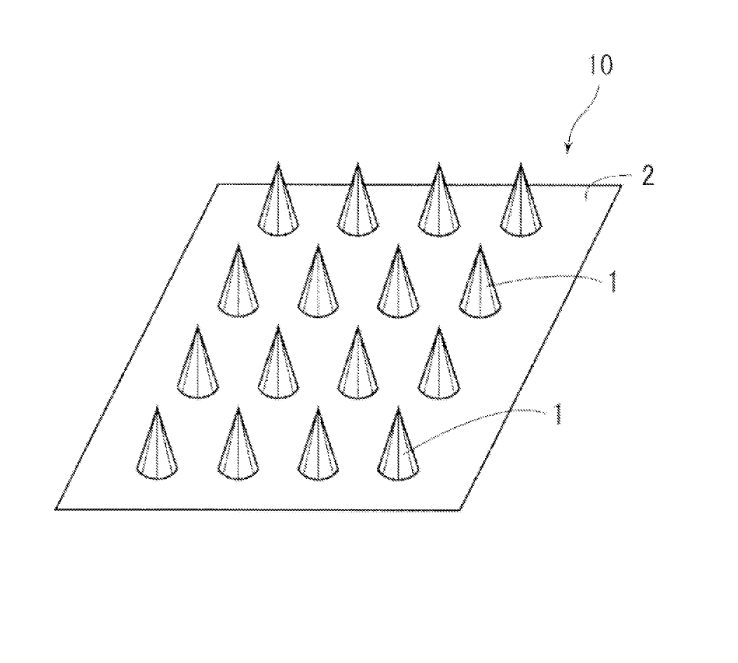

### CROSS-REFERENCE TO RELATED APPLICATIONS

The present application claims priority from U.S. Provisional Application No. 61/665,974, filed Jun. 29, 2012.

### FIELD OF THE INVENTION

This invention is related to dissolvable microneedle arrays comprising cosmetic ingredients for insertion into skin.

### BACKGROUND OF THE INVENTION

It is well known that in order to provide decorating and/or functional effect cosmetic formulations such as solutions, ointments, creams, tapes, patches are commonly administered.

These formulations should be applied to the skin. Thus, they are readily lost or removed under various conditions, such as perspiration, washing and external pressure. Further, they need the permeation of cosmetic actives into skin obtain optimal efficacy. However, it is difficult deliver optimal efficacy of many cosmetic actives due to the barrier properties of skin which prohibits the entrance of foreign materials.

Recently, to solve these problems, microneedles fabricated of metal or plastic coated with cosmetic agents on their surface have been used as an approach to actually deliver cosmetic agents to a desired site of skin. However, with this approach, a small quantity of cosmetic agents can be administrated, and there is a risk that solid microneedle fragments will remain in skin. Therefore, their use raises many safety concerns.

Based on the foregoing, it is clear that there is a need for a microneedle array which is capable of delivering cosmetic ingredients into the skin without safety risks associated with metal or plastic microneedles. One approach to serve this need involves the design of microneedle arrays having water-soluble microneedles. For example, polysaccharides and starches have been formed into water-soluble microneedles.

When the above functional microneedle is inserted into skin, cosmetic agents are delivered to the skin with dissolution, swelling or breaking off of the needle in skin. Thus, it is possible to actually deliver cosmetic agents to a desired site of skin with the functional microneedle. As the needle is very sharp, there is no pain or bleeding caused by the insertion of needle into skin, and further, the closure of the pores is rapid. Therefore, the functional microneedle is suitable to actually deliver cosmetic agents to a desired site of skin.

If the needle is lack in mechanical strength, it would be broken during the insertion and unable to be inserted into skin. Thus, the needle preferably has sufficient mechanical strength. However, it is difficult to fabricate the needle with suitable mechanical strength by the use of saccharide such as maltose that can dissolve and disappear in the body. Moreover, maltose has a disadvantage of poor practicability due to its high hydroscopicity.

Hyaluronic acid in combination with biopolymers, such as chitin, has been developed to form a rigid and water-soluble material for forming microneedles. Cosmetic and other actives may be incorporated into these formulations to deliver various benefits to skin. But the level of benefit agents in the microneedles is limited by the need to maintain rigidity of the needles by maintaining a critical mass of the biopolymer, and hyaluronic acid having a molecular weight of about 400,000. These narrow formulation parameters restrict variation among suitable compositions to incorporate into the microneedles.

Therefore, there is an ongoing need for a microneedle array which is capable of delivering a wide range of benefit agents to skin, while eliminating risks associated with plastic and metal microneedles. And the microneedle array should avoid the drawbacks of hyaluronic acid and biopolymer-based microneedles.

### SUMMARY OF THE INVENTION

The purpose of the present invention is to provide a microneedle array which can be easily inserted into skin, leave contained cosmetic agents under the surface of skin by dissolution, swell or break off of needles, and dissolve or disappear into skin. It is another object of the present invention to provide a microneedle array for the administration of soluble and/or in-soluble cosmetic agents to skin.

The present invention provides a microneedle array which comprises a substrate and cone-shaped or pyramid-shaped microneedles for skin insertion fixed on the substrate. The microneedles are readily dissolved after puncturing the outer surface of human skin.

### BRIEF DESCRIPTION OF THE DRAWINGS

The lone FIGURE is a schematic view of the microneedle array.

### DETAILED DESCRIPTION OF THE INVENTION

The microneedle array of the present invention comprises a substrate and cone-shaped or pyramid-shaped microneedles for skin insertion on the substrate. The microneedles are comprised of one or more water soluble materials. For example, U.S. Patent Publication 2010/0228203 to Quan et al. discloses chitosan, collagen and gelatin, as a material which can dissolve or swell in the body. Other suitable materials include polysaccharides such as maltose, alginate and agarose, cellulose such as carboxymethylcellulose and hydroxypropylcellulose, starch. Particularly preferred, is an embodiment containing over 50 weight percent of hyaluronic acid.

Hyaluronic acid used in the present invention is a kind of glycosaminoglycan. Hyaluronic acid is composed of the repeating disaccharide unit of N-acetylglucosamine and glucuronic acid. Glycosaminoglycan is also called mucopolysaccharide. It is preferable to use the hyaluronic acid that obtained from organism such as crista galli and umbilical cord and that obtained with the with cultivation of lactic acid bacteria, streptococcus.

The microneedle product from hyaluronic acid becomes harder as the weight average molecular weight of hyaluronic acid is smaller. And its mechanical strength increases with the weight average molecular weight of hyaluronic acid. Thus, when the weight average molecular weight of hyaluronic acid as raw material is smaller, the microneedles for skin insertion become harder and it is easy to insert them into skin, while their mechanical strength decrease and the needles are easily broken during storage and insertion. For this reason, it is preferable to use the hyaluronic acid with the weight average molecular weight larger than 400,000. The weight average molecular weight is determined by the method of gel permeation chromatography.

In one form of the invention, the microneedles for skin insertion contain over 50 weight percent of hyaluronic acid and they are can be dissolve or swell in the body. So far as the microneedles for skin insertion contain over 50 weight percent of mentioned biomaterials, other biomaterials of less than 50 weight percent which can dissolve or swell in the body can be used. Hyaluronic acid has the cosmetic result of bulging skin. Thus, it is preferred to only use hyaluronic acid to fabricate the microneedles for skin insertion.

Besides hyaluronic acid, as a material which can dissolve or swell in the body, for example, are polysaccharides such as maltose, alginate and agarose, cellulose derivatives such as carboxymethylcellulose and hydroxypropylcellulose, starch.

In one form of the invention, the microneedles for skin insertion are characterized by containing 50 to 70 weight percent of collagen and 50 to 30 weight percent of hyaluronic acid.

The microneedles for skin insertion which contain 50 to 70 weight percent of collagen and 50 to 30 weight percent of hyaluronic acid, which are dissolvable and swellable in the body, have suitable mechanical strength and are easy to insert into skin and have good solubility in the skin. Thus, it is preferred that the microneedles for skin insertion is composed of biomaterials of 50 to 70 weight percent of collagen and 50 to 30 weight percent of hyaluronic acid which can dissolve or swell in the body.

In an even more preferred embodiment, the microneedles are comprised of a dissolvable shell. And the shell contains one or more cosmetic actives which may or may not be water soluble. For example, the dissolvable shell is comprised of a water-soluble polyvinyl alcohol (PVA) material. The PVA shell is of sufficient rigidity to be formed into a microneedle which is sufficiently sharp to puncture the surface of human skin. Such PVA materials are available from, for example, BDP Green Technology Corp. In a preferred embodiment, the PVA material is biodegradable.

The microneedle shell is not necessarily limited to water-soluble materials depending on the site of entry for the microneedles. For example, if the needles are intended to puncture, and dissolve into, a fatty region, it may be appropriate to form the microneedle shell from, at least in part, a hydropobic or hydrophobically-modified material, liposomes, powders, and the like.

In one embodiment, the microneedles comprise a PVA shell which encapsulates one or more cosmetic agents. The cosmetic agents may be water-soluble or water-insoluble as further described herein. The cosmetic agents may also be liquid and/or in aqueous form. If the cosmetic agents are aqueous, then the PVA material should be constructed with sufficient saponification to maintain relative stability prior to penetration of human skin. Alternatively, the PVA microneedles comprising an aqueous composition should be stored at a sufficiently cool temperature to maintain stability prior to penetration of human skin.

The FIGURE is a general view of the microneedle array 10. As shown in the FIGURE, a plurality of microneedles 1 for skin insertion are attached or integrally formed to the substrate 2.

The microneedles 1 for skin insertion are necessary to be penetrated into skin. Moreover, the tops of the microneedles 1 inserted into skin are essential to remain in skin with dissolution, swelling and breaking off in the skin. Thus, the microneedles 1 for skin insertion become gradually finer from the base to the top and are preferred to have sharp tops. In detail, the shape of the microneedles 1 is preferred to be circular cone or polygonal pyramid such as triangulate pyramid, quadrangular pyramid, hexagonal pyramid and octagonal pyramid.

The diameter or the length of one side to another at the base of the microneedles 1 for skin insertion is preferred to be 100 to 300 μm. The height of the microneedles 1 for skin insertion is preferred to be 100 to 1200 μm. The space between microneedles 1 is not specially defined and is preferred generally to be 100 to 1000 μm.

A variety of cosmetic ingredients may be delivered as components of the dissolvable microneedle array herein. For example, whitening ingredients, antiwrinkle ingredients, blood circulation promotion ingredients, dietary aid, antibacterial agents; vitamins may be included in the microneedle compositions.

As whitening ingredients, for example, are vitamin C and derivatives such as ascorbyl glucoside, ascorbyl palmitate, licorice extract, yeast extracts, trametes, aspergillus, exophilia, resveratrol and derivatives resveratrol phosphate, resveratrol ferulate, oxyresveratrol, ferulic acid and its derivatives, kojic acid, ellagic acid, hinokitiol, soybean extracts, scutellaria extract, mulberry extract, molasses, tetrahydrocurcumins, glycyrrhetinic acid, pomegranate, grape seed extract, viapure hops, BV-OSC—tetrahexyldecylascorbate, ascorbic acid disodium phosphate, ascorbic acid glucoside, α(β)-arbutin, ascorbyl palmitate, resorcinol, and tranexamic acid.

As antiwrinkle ingredients, for example, are retinol, tretinoin, retinol acetate, vitamin A palmitate. As blood circulation promotion ingredients, for example, are tocopheryl acetate, and capsacin. As dietary aids, for example, are centella asiatica, chlorella extract, boswellia extract, whey protein, ursolic acid, white birch, biopeptide EL—palmitoyloligopeptide, pycnogenol, zincidone, siegesbeckia, silymarin, argireline, dill extract, NAB fennel seed extract, anogeissus bark extract, viapure menyanthes, tetrahexyldecylascorbate, aminopropylascorbylphosphate, yeast ferments, phytomatrix, N-acetyl glucosamine, urea, resveratrol and derivatives its derivatives, raspberry ketone, evening primrose, and seaweed extract.

As antibacterial agents, for example, are isopropylmethylphenol, photosensitizers, zinc oxide. As vitamins, for example, are vitamin D2, vitamin D3, vitamin K. Other suitable cosmetic ingredients include roxisome, photosome, ultrasomes, growth factors, RNA, DNA fragments, genes, hyaluronic acid, and salicylic acid.

Although either of the mentioned cosmetic agents has the molecular weight of less than 600, the one has high molecular weight also can be used. As preferable cosmetic agents that have high molecular weight, for example, are bioactive peptide and its derivative, nucleinic acid, oligonucleotide, various kinds of antigens, bacteria, virus fragment.

As a bioactive peptide and its derivative, for example, are calcitonin, adrenocorticotropic hormone, parathormone (PTH), hPTH (1→34), EGF, insulin, secretin, oxytocin, angiotensin, β-endorphin, glucagon, vasopressin, somatostatin, gastrin, luteinizing hormone-releasing hormone, enkephalin, neurotensin, atrial natriuretic peptide, somatotropin, somatotropin-releasing hormone, bradykinin, substance P, dynorphin, thyroid stimulating hormone, mammotrophic hormone, interferon, interleukin, G-C SF, glutathione peroxidase, superoxide dismutase, desmopressin, somatomedin, endothelin, placenta extract and salts of them. As antigens, for example, are HBs surface antigen, HBe antigen, tetanus toxoid, diphtheria toxoid, amyloidβprotein.

As mentioned above, in the microneedle array 10, a plurality of microneedles 1 inserted into skin are fixed on the substrate 2. The substrate 2, which the microneedles 1 inserted into skin can be formed on, is not specially defined to have affinity of attachment with the microneedles 1 inserted into skin. The substrate 2 can be a film or sheet made of materials such urethane resin, polyvinyl alcohol and aluminum. The thickness of the substrate 2 can be, for example, 100 to 1000 μm. In addition, the substrate 2 can be fabricated of materials that can dissolve or swell in the body like the microneedles 1 inserted into skin.

The method of manufacturing of the microneedle array 10 is not specially limited. The microneedle array 10 can be fabricated by any well-known method, such as the following method (1) to (4).

In the method (1), the dissolvable shell material, and if necessary, the cosmetic agents, are put on a mold in which the holes corresponding to the microneedle shapes 1 have been patterned. Then dry the solution at room temperature or by heating to evaporate water. After laminating the substrate 2 to the needles, the microneedles 1 for skin insertion and substrate 2 are obtained by peeling them off from the mold.

In the method (2), the solution described above is put on the mold mentioned above to form a substrate layer on the mold and microneedles in the mold. After evaporating the water of the solution at room temperature or by heating, the microneedle array is obtained by peeling the substrate off from the mold.

According to the method (2), can be obtained the microneedle array 10, of which the substrate 2 and the microneedles 1 for skin insertion are both fabricated. The above microneedles contain over 50 weight percent of biomaterials chosen from chitosan, collagen, gelatin, hyaluronic acid which can dissolve or swell in the body, and can contain cosmetic agents according to the requirement.

In the method (3), the solution which contains over 50 weight percent of biomaterials chosen from chitosan, collagen, gelatin, hyaluronic acid, materials that can dissolve or swell in the body, and cosmetic agents according to the requirement; is injected as the microneedles 1 for skin insertion onto the substrate 2. And then the microneedle array is obtained by drying the solution at room temperature or by heating.

In the mentioned methods of manufacturing, as a material of the microneedles 1 for skin insertion, the needles can be composed of over 50 weight percent of the biomaterials chosen from chitosan, collagen, gelatin, and other materials those can dissolve or swell in the body. It is preferred to use collagen from biological origin with 50 to 70 weight percent and hyaluronic acid from biological origin with 50 to 30 weight percent and other biomaterials dissolve or swell in the body.

The composition of the microneedle array 10 is as mentioned above. The microneedles 1 for skin insertion of the microneedle array 10 have suitable mechanical strength, toughness and hardness, and can be easily inserted into skin without breaking followed by dissolving and disappearing in the skin.

Therefore, it is possible to actually deliver the biomaterials chosen from chitosan, collagen, gelatin or hyaluronic acid to the desired part of skin. It is also possible to deliver cosmetic agents to the desired part of skin by adding them in the microneedles 1 for skin insertion. In addition, the microneedles 1 for skin insertion are able to contain a great quantity of cosmetic agents if cosmetic agents are water soluble. Moreover, it is not necessary to fabricate the microneedle array by heating when soluble biomaterials are used as the material of the microneedles for skin insertion. In this way, the decrease in the effect of cosmetic agents due to heat decomposition can be avoided.

What is claimed is:

1. An applicator for applying cosmetic agents into human skin, comprising: (a) a base, (b) a plurality of dissolvable microneedles fixed to said base and projecting therefrom a distance sufficient to penetrate into the skin, said microneedles being made of a material that is capable of disintegration and dispersion into the skin, (c) a cosmetic agent carried by said microneedles for delivery by said microneedles into the skin, and (d) wherein said dissolvable microneedles are configured as a dissolvable exterior shell and said shell encapsulates one or more cosmetic agents.

2. An applicator according to claim 1, wherein said dissolvable exterior shell is formed substantially from a water-soluble polyvinyl alcohol (PVA) material.

3. An applicator according to claim 2 wherein said cosmetic agent is comprised in an aqueous composition.

4. An applicator according to claim 1 wherein said cosmetic agent is dissolvable exterior shell is formed substantially from a material selected from the group consisting of hydrophobic materials, hydrophobically-modified materials, and liposomes.

5. An applicator according to claim 1 wherein said base and said microneedles are integrally molded from the same material.

6. An applicator according to claim 1 wherein said microneedles are generally cone shaped.

7. An applicator according to claim 1 wherein said microneedles are square in cross-section.

8. An applicator according to claim 1 wherein said microneedles are polygonal in cross-section.

9. An applicator according to claim 1 and wherein said microneedles are at least partially elliptical in cross-section.

10. An applicator according to claim 1 wherein said microneedles are constricted intermediate their ends to facilitate breaking off the portions of the needles beyond the narrow portions to leave those portions in the skin.

11. An applicator according to claim 1 wherein said microneedles have relatively thin exterior shell and relatively thick encapsulated cosmetic agent region, said base with a step between said portions to facilitate separation of said exterior shells from said encapsulated cosmetic agent regions, with the exterior shells remaining in the skin

12. An applicator according to claim 1 wherein said microneedles have tips which are knife-shaped to facilitate insertion into the skin.

13. An applicator according to claim 1, wherein said microneedles comprise at least one water-insoluble benefit agent.

14. An applicator according to claim 15, wherein said water-insoluble benefit agent is selected from the group consisting of lipids, oils, waxes, proteins, hydrophobically surface-modified pigments, inorganic compounds, and mixtures thereof.
